(19) United States
(12) Patent Application Publication
(10) Pub. No.: US 2021/0007957 A1
(43) Pub. Date: Jan. 14, 2021

(54) PROCESS FOR CURL-RELAXING AND/OR STRAIGHTENING KERATIN FIBRES, USING A THIOL-BASED REDUCING AGENT AND A DICARBOXYLIC ACID DERIVATIVE, AND STRAIGHTENING KIT

(71) Applicant: L'OREAL, Paris (FR)

(72) Inventors: Christian BLAISE, AULNAY-SOUS-BOIS (FR); Henri SAMAIN, CHEVILLY LARUE (FR)

(21) Appl. No.: 17/043,000

(22) PCT Filed: Apr. 8, 2019
(86) PCT No.: PCT/EP2019/058795

§ 371 Date: Sep. 29, 2020

(30) Foreign Application Priority Data
Apr. 9, 2018 (FR) 1853064

(51) Int. Cl. A61K 8/46; A61K 8/36; A61Q 5/04

(52) U.S. Cl. CPC A61K 8/46; A61Q 5/04; A61K 8/36

(57) ABSTRACT

Methods and compositions for relaxing and/or straightening keratin fibres are disclosed. The method comprises i) simultaneously or sequentially applying to the keratin fibres a composition (A) and a composition (B); or ii) alternatively, applying to the keratin fibres a composition (D); and iii) heating the keratin fibres with a heating tool after both the composition (A) and the composition (B) are applied to the keratin fibres, or after the composition (D) is applied to the keratin fibres. The composition (A) is acidic and comprises at least one thiol-based reducing agent. The composition (B) comprises at least one compound chosen from compounds of formula (I): RO—C(O)-ALK-C(O)—OR′. The composition (D) is acidic and comprises at least one thiol-based reducing agent and at least one compound chosen from the compounds of formula (I).

### CROSS REFERENCE TO RELATED APPLICATION

This is a national stage application of PCT/EP2019/058795, filed internationally on Apr. 8, 2019, which claims priority to French Application No. 1853064, filed on Apr. 9, 2018, both of which are incorporated by reference herein in their entireties.

The present invention relates to a process for curl-relaxing and/or straightening keratin fibres, such as the hair, which comprises the application to the fibres of at least one composition comprising one or more thiol-based reducing agents in an amount of less than 8% by weight relative to the weight of the composition which comprises it, of at least one composition comprising comprising one or more dicarboxylic acids and a step of heat treatment of the fibres by means of a heating tool.

The invention also relates to the use of the composition(s) comprising reducing agents and of the composition(s) comprising dicarboxylic acids or derivatives in a process for curl-relaxing and/or straightening keratin fibres.

Finally, the invention relates to a multi-compartment device or “kit” that is suitable for performing such a process.

Many people are not satisfied with the appearance of their hair. In particular, people who have curly hair usually seek to obtain straight hair.

To obtain permanent reshaping of the hair such as straightening of the hair, uncurling or relaxing of the curls, the technique most commonly used consists, in a first stage, in opening the —S—S— disulfide bonds of keratin (keratocystine) generally by means of a basic composition containing a sulfur-based reducing agent (reduction step), and then, after having rinsed the head of hair thus treated, generally with water, in reconstituting, in a second stage, said disulfide bonds by applying to the hair, which has been placed under tension beforehand, an oxidizing composition (oxidation step, also known as the fixing step) so as finally to give the hair the desired shape.

The new shape given to the hair by such a chemical treatment is eminently long-lasting and especially withstands washing with water or shampoos, as opposed to the simple standard techniques of temporary reshaping, such as hairsetting.

Many products intended for straightening or uncurling the hair or for curl-relaxing exist on the market.

The products intended for straightening or uncurling are generally formulated either using very alkaline compositions, with a pH above 12, or using a high concentration of thiols, such as mercaptan compounds.

The oxidizing compositions required for performing the fixing step are usually compositions based on aqueous hydrogen peroxide solution.

However, the application of these products is generally long, with a longer or shorter leave-on time depending on the product, the type of hair and the desired effect. It requires precise know-how, which is mainly due to the high contents of reducing agents used in the reducing compositions or to the high contents of hydroxides and/or to the very alkaline pH of the relaxing compositions, and also to the various longer or shorter leave-on times of these compositions.

It has also been found that the use of these reducing agents or of these strong alkaline agents can lead to scalp discomfort (irritation, itching, etc.).

Moreover, the compositions employed often pose problems of odours, in particular the reducing compositions and especially those containing thiols. Hair treated with these compositions may also retain an unpleasant odour.

Furthermore, the reducing agents are generally used in high concentrations, which may lead to more or less pronounced degradation of the keratin fibre, in particular when the hair is dyed.

These techniques can thus bring about, in the long term, impairment of the quality of the hair, leading to a decrease in its cosmetic properties, such as its vitality or its sheen, and a degradation in its mechanical properties, more particularly in its mechanical strength.

The impact of these treatments on the quality of the head of hair is poorly tolerated by users, who then have a tendency to abandon their treatments or to perform them at long intervals.

Furthermore, the use of a curl-relaxing treatment is especially intricate if the quality of the hair comprises disparities along the fibre, as in the case of damaged ends (which occurs in particular in the case of long hair) or in the case of bleached parts of the hair (which occurs in particular in highlighted hair).

One possibility is to use compositions containing more or less concentrated thiols on the different parts of the head of hair as a function of its state, which would necessitate lock-by-lock manipulation. However, the application of a thiol-based composition is unpleasant for the operator (typically the hair stylist), who then has a tendency to go faster during this application, and does not wish to perform these lock-by-lock manipulations.

Another possibility is to apply a single aqueous thiol formulation, in general in a concentration generally greater than 0.5 M, to the head of hair and then to treat the locks with the heating tool while limiting the contact of the tool with the embrittled areas of the fibres, or while reducing the temperature of said tool.

However, this solution is not satisfactory either, since the embrittled parts of the hair, which will have been manipulated with the heating tool for a reduced contact time or at a reduced temperature, will be sparingly treated and will return to curling before the other hair.

Thus, a process is sought that can be used in the case of heterogeneous hair, making it possible to relax the curls and giving an equivalent straightening effect irrespective of the initial state of the keratin fibre.

FR 3 009 785 discloses a hair cosmetic composition, in particular for Brazilian type straightening, comprising a keratin solution, preferably a keratin hydrolysate, at least one acidic catalyst and a carbonyl binding compound, characterized in that the carbonyl binding compound is urea or a derivative of urea.

The term “heterogeneous hair” is intended to mean hair comprising, along the keratin fibres of the hair, embrittled and/or damaged areas, and dry, coarse, brittle or split areas.

In parallel, this process must retain good performance qualities in terms of the straightening of keratin fibres. In particular, the obtained result should be persistent on shampooing several times.

Furthermore, it is desirable for these treatments to be easy to apply, and comfortable for the operator to use, in particular in terms of odours deriving from the use of such a process.

The Applicant has now discovered that the use of a particular process in at least two steps makes it possible to meet the abovementioned objectives.

It has thus discovered, surprisingly, a process for curl-relaxing and/or straightening keratin fibres, in particular human keratin fibres such as the hair, in several steps, which comprises:

either:

i) the application to said fibres of an acidic composition (A), preferably having a pH of between 1 and 5 inclusive, and containing one or more thiol-based reducing agents in an amount of less than 8% by weight relative to the total weight of the composition (A), said reducing agent(s) preferably comprising one or more carboxylic groups;

ii) the application to said fibres of a distinct composition (B) containing one or more dicarboxylic acids or derivatives of formula (I): RO—C(O)-ALK-C(O)—OR′ and also mineral or organic salts thereof, in which ALK represents a linear or branched (C₁-C₆)alkylene group, R and R′, which may be identical or different, represent a hydrogen atom or an optionally substituted (C₁-C₆)alkyl group, an optionally substituted (C₂-C₆)alkenyl group, an optionally substituted (hetero)aryl(C₁-C₄)alkyl group, such as benzyl, an optionally substituted (hetero)cyclic(C₁-C₄)alkyl group, such as cyclohexylmethyl, or an optionally substituted (hetero)aryl group such as phenyl, preferably R and R′ represent a hydrogen atom;

or:

iii) the application of an acidic composition (D), preferably having a pH of between 1 and 5 inclusive, and containing:

a) one or more thiol-based reducing agents in an amount of less than 8% by weight relative to the total weight of the composition (D), said reducing agent(s) preferably comprising one or more carboxylic groups; and

b) one or more dicarboxylic acids of formula (I) as defined previously; followed

iv) by a step of heat treatment of the fibres by means of a heating tool; it being understood that:
steps i) and ii) may be applied together or separately, preferably together,
steps i) and ii) are followed by heat treatment step iv) or else step iii) is followed by heat treatment step iv).
   This process makes it possible to achieve the desired properties, inter alia in terms of integrity, quality and cosmeticity of the keratin fibres, while at the same time obtaining curl relaxation and/or straightening of keratin fibres that is of good quality and long-lasting in particular on embrittled hair, this being despite a very small amount of thiol-based reducing agent.
   Another subject of the invention is a composition (D) preferably having a pH of between 1 and 5 inclusive, and containing:

a) one or more thiol-based reducing agents in an amount of less than 8% by weight relative to the total weight of the composition (D), said reducing agent(s) preferably comprising one or more carboxylic groups; and

b) one or more dicarboxylic acids or derivatives of formula (I) as defined previously.

One subject of the present invention is thus a process for curl-relaxing and/or straightening keratin fibres, in particular keratin fibres such as the hair, comprising steps either i), ii) and then iv) or iii) and then iv) as defined previously.

According to one advantageous variant of the invention, steps i) and ii) are applied simultaneously to the keratin fibres.

More preferentially, the process of the invention carries out iii) the application of the acidic composition (D), as defined previously; followed by iv) a step of heat treatment of the fibres by means of a heating tool.

The implementation of this process makes it possible to obtain curl relaxation and/or straightening of keratin fibres that is of good quality and persistent on shampooing several times while preserving the quality and integrity of the keratin fibres. Furthermore, the implementation of this process makes it possible to provide the keratin fibres with good cosmetic properties, in particular sheen and a soft feel.

In addition, the process of the invention makes it possible to transform the shape of the hair without risk in the case where several successive applications would be carried out.

The invention thus makes it possible to transform the shape of the hair by carrying out successive applications without causing strong damage to the fibre.

In one preferred variant, the process according to the invention is a process for straightening keratin fibres, in particular the hair.

A subject of the invention is also a kit that is suitable for performing the process of the invention. This kit comprises at least two compartments:

a first compartment comprising a composition (A) as defined previously; and
a second compartment comprising a composition (B) as defined previously.

A subject of the present invention is also a composition (A) or (D) that is suitable for carrying out the process of the invention. These cosmetic compositions comprise one or more thiol-based reducing agents and are acidic. They may have a pH of between 1 and 5 inclusive, preferably between 2.5 and 4, such as pH=3.5. Composition (B) may be acidic and have a pH of between 1 and 5, preferably between 2.5 and 4, such as pH=3.5.

The term “pH of between 1 and 5 inclusive” is intended to mean that the limits 1 and 5 are included in the pH range.

A subject of the present invention is also the use of compositions (A) and (B) or (D) as defined previously, in a process for shaping, curl-relaxing and/or straightening keratin fibres, especially human keratin fibres such as the hair.

Such a process is rapid and simple to perform, and does not require any particular know-how.

Furthermore, the implementation of this process makes it possible to obtain a relaxing of curls and/or a reduction of the volume of the head of hair, which is gradual. By virtue of its protection of the integrity of the keratin fibres, the implementation of this process makes it possible to give them good cosmetic properties, in particular of sheen and softness to the touch.

In particular, it is possible, by virtue of the invention, to modulate the desired effect, by successive implementations of such a process, either using treatments with the same concentrations of active agents and the same conditions (temperature, leave-on-time, etc.), or by varying the concentrations of active agents and/or the conditions, from one application to the other. Finally, the process according to the invention makes it possible to substantially reduce the unpleasant odours derived from performing a standard curl relaxation and/or straightening process.

Other characteristics and advantages of the invention will emerge more clearly on reading the description, the examples and the figures that follow.

For the purposes of the present invention and unless otherwise indicated:

the term “(hetero)aryl” is intended to mean aryl and heteroaryl groups;
the “aryl” or “heteroaryl” radicals or the aryl or heteroaryl part of a radical may be substituted with at least one substituent borne by a carbon atom, chosen from:

a C₁-C₆ and preferably C₁-C₄ alkyl radical optionally substituted with one or more radicals chosen from the radicals hydroxyl, C₁-C₂ alkoxy, C₂-C₄ (poly)hydroxyalkoxy, acylamino, amino substituted with two C₁-C₄ alkyl radicals, which may be identical or different, optionally bearing at least one hydroxyl group, or the two radicals possibly forming, with the nitrogen atom to which they are attached, a saturated or unsaturated, optionally substituted 5- to 7-membered and preferably 5- or 6-membered heterocycle optionally comprising another nitrogen or non-nitrogen heteroatom;

a halogen atom;

a hydroxyl or thiol group;

a C₁-C₆ alkoxy or C₁-C₆ alkylthio radical;

a (poly)hydroxy(C₂-C₆)alkoxy radical;

an amino radical;

a 5- or 6-membered heterocycloalkyl radical, preferentially morpholino, piperazino, piperidino or pyrolidino, which is optionally substituted with a (C₁-C₄) alkyl radical, preferentially methyl;

a 5- or 6-membered heteroaryl radical, preferentially imidazolyl, optionally substituted with a (C₁-C₄)alkyl radical, preferentially methyl;

an amino radical substituted with one or two identical or different C₁-C₆ alkyl radicals, optionally bearing at least:
i) a hydroxyl group,
ii) an amino group optionally substituted with one or two optionally substituted C₁-C₃ alkyl radicals, said alkyl radicals possibly forming with the nitrogen atom to which they are attached a saturated or unsaturated, optionally substituted 5- to 7-membered heterocycle, optionally comprising at least one other nitrogen or non-nitrogen heteroatom,
iii) a quaternary ammonium group —N⁺R′R″R′″, M⁻ for which R′, R″ and R″′, which may be identical or different, represent a hydrogen atom or a C₁-C₄ alkyl group and M⁻ represents an anionic counterion,
iv) or an optionally cationic 5- or 6-membered heteroaryl radical, preferentially imidazolium, optionally substituted with a (C₁-C₄)alkyl radical, preferentially methyl;

an acylamino radical (—N(R)—C(O)—R′) in which the R radical is a hydrogen atom or a C₁-C₄ alkyl radical optionally bearing at least one hydroxyl group and the R′ radical is a C₁-C₂ alkyl radical;

a carbamoyl radical ((R)₂N—C(O)—) in which the R radicals, which may be identical or different, represent a hydrogen atom or a C₁-C₄ alkyl radical optionally bearing at least one hydroxyl group;

an alkylsulfonylamino radical (R′—S(O)₂—N(R)—) in which the R radical represents a hydrogen atom or a C₁-C₄ alkyl radical optionally bearing at least one hydroxyl group and the R′ radical represents a C₁-C₄ alkyl radical, or a phenyl radical;

an amino sulfonyl radical ((R)₂N—S(O)₂—) in which the R radicals, which may be identical or different, represent a hydrogen atom or a C₁-C₄ alkyl radical optionally bearing at least one hydroxyl group;

a carboxyl radical in the acid or salified form (preferably salified with an alkali metal or a substituted or unsubstituted ammonium);

a cyano group;

a nitro or nitroso group;

a polyhaloalkyl group, preferably trifluoromethyl;

the cyclic or heterocyclic part of a non-aromatic radical may be substituted with at least one substituent chosen from the following groups:

hydroxyl;

C₁-C₄ alkoxy, C₂-C₄ (poly)hydroxyalkoxy;

C₁-C₄ alkyl;

alkylcarbonylamino (R—C(O)—N(R′)—) in which the radical R′ is a hydrogen atom or a C₁ -C₄ alkyl radical optionally bearing at least one hydroxyl group, and the radical R is a C₁-C₂ alkyl radical or an amino radical optionally substituted with one or two C₁-C₄ alkyl groups, which may be identical or different, themselves optionally bearing at least one hydroxyl group, said alkyl radicals possibly forming, with the nitrogen atom to which they are attached, a saturated or unsaturated, optionally substituted 5- to 7-membered heterocycle optionally comprising at least one other nitrogen or non-nitrogen heteroatom;

alkylcarbonyloxy (R—C(O)—O—) in which the radical R is a C₁-C₄ alkyl radical or an amino group optionally substituted with one or two identical or different C₁-C₄ alkyl groups themselves optionally bearing at least one hydroxyl group, said alkyl radicals possibly forming with the nitrogen atom to which they are attached a saturated or unsaturated, optionally substituted 5- to 7-membered heterocycle, optionally comprising at least one other nitrogen or non-nitrogen heteroatom;

alkoxycarbonyl (R—X—C(O)—) in which the radical R is a C₁-C₄ alkoxy radical, X is an oxygen atom or an amino group optionally substituted with a C₁-C₄ alkyl group itself optionally bearing at least one hydroxyl group, said alkyl radical possibly forming with the nitrogen atom to which it is attached a saturated or unsaturated, optionally substituted 5- to 7-membered heterocycle, optionally comprising at least one other nitrogen or non-nitrogen heteroatom;

a carbocyclic or heterocyclic radical, or a non-aromatic part of an aryl or heteroaryl radical, may also be substituted with one or more oxo groups;
a hydrocarbon-based chain is unsaturated when it comprises one or more double bonds and/or one or more triple bonds;
an “aryl” radical represents a monocyclic or fused or non-fused polycyclic carbon-based group, comprising from 6 to 22 carbon atoms, at least one ring of which is aromatic; preferentially, the aryl radical is a phenyl, biphenyl, naphthyl, indenyl, anthracenyl or tetrahydronaphthyl;
a “heteroaryl radical” represents a fused or non-fused, optionally cationic, 5- to 22-membered monocyclic or polycyclic group, comprising from 1 to 6 heteroatoms chosen from nitrogen, oxygen, sulfur and selenium atoms, and at least one ring of which is aromatic; preferentially, a heteroaryl radical is chosen from acridinyl, benzimidazolyl, benzobistriazolyl, benzopyrazolyl, benzopyridazinyl, benzoquinolyl, benzothiazolyl, benzotriazolyl, benzoxazolyl, pyridyl, tetrazolyl, dihydrothiazolyl, imidazopyridyl, imidazolyl, indolyl, isoquinolyl, naphthoimidazolyl, naphthooxazolyl, naphthopyrazolyl, oxadiazolyl, oxazolyl, oxazolopyridyl, phenazinyl, phenoxazolyl, pyrazinyl, pyrazolyl, pyrilyl, pyrazoyltriazyl, pyridyl, pyridinoimidazolyl, pyrrolyl, quinolyl, tetrazolyl, thiadiazolyl, thiazolyl, thiazolopyridyl, thiazoylimidazolyl, thiopyrylyl, triazolyl, xanthinyl or thioxanthinyl;
a “heterocyclic radical” is a fused or non-fused, 5- to 22-membered monocyclic or polycyclic radical, possibly containing one or two unsaturations but non-aromatic, comprising from 1 to 6 heteroatoms chosen from nitrogen, oxygen, sulfur and selenium atoms;
a “carbocyclic radical” is a monocyclic or polycyclic, fused or non-fused, 5- to 22-membered radical which may contain one or two unsaturations but is non-aromatic, such as cyclobutyl, cyclopentyl or cyclohexyl;
a “(hetero)cyclic radical” is a heterocyclic or carbocyclic radical;
an “alkyl” radical is a linear or branched, saturated C₁-C₁₀, in particular C₁-C₈, more particularly C₁-C₆ and preferably C₁-C_{4,} hydrocarbon-based radical;
an “alkoxy” radical is an “alkyl-oxy” radical in which the alkyl group is as defined previously;
an “alkenyl” radical is a linear or branched C₂-C₁₀, in particular C₂-C₈, more particularly C₂-C₆, preferably C₂-C₄, hydrocarbon-based radical comprising one or more conjugated or non-conjugated unsaturations, preferably comprising one or two double bonds, such as ethylenyl;
the expression “optionally substituted” attributed to the alkyl or alkenyl radical is intended to mean that said alkyl or alkenyl radical may be substituted with one or more radicals chosen from the following radicals: i) hydroxyl, ii) C₁-C₄ alkoxy, iii) acylamino, iv) amino optionally substituted with one or two identical or different C₁-C₄ alkyl radicals, said alkyl radicals possibly forming, with the nitrogen atom that bears them, a 5- to 7-membered heterocycle, optionally comprising another nitrogen or non-nitrogen heteroatom; v) carboxy; or vi) aryl such as phenyl optionally substituted with one or more (di)(C₁-C₄)(alkyl)amino groups or hydroxyl groups;
an “alkoxy” radical is an alkyl-oxy radical for which the alkyl radical is a linear or branched C₁-C₈ and preferentially C₁-C₆ hydrocarbon-based radical;
when the alkoxy group is optionally substituted, this implies that the alkyl group is optionally substituted as defined above;
the term “organic or mineral acid salt” is more particularly intended to mean salts chosen from a salt derived from i) hydrochloric acid HCl, ii) hydrobromic acid HBr, iii) sulfuric acid H₂SO₄, iv) alkylsulfonic acids: Alk-S(O)₂OH such as methylsulfonic acid and ethylsulfonic acid; v) arylsulfonic acids: Ar—S(O)₂OH such as benzenesulfonic acid and toluenesulfonic acid; vi) citric acid; vii) succinic acid; viii) tartaric acid; ix) lactic acid; x) alkoxysulfinic acids: Alk-O-S(O)-OH such as methoxysulfinic acid and ethoxysulfinic acid; xi) aryloxysulfinic acids such as tolueneoxysulfinic acid and phenoxysulfinic acid; xii) phosphoric acid H₃PO₄; xiii) acetic acid CH₃C(O)—OH; xiv) triflic acid CF₃SO₃H; and xv) tetrafluoroboric acid HBF₄;
the term “anionic counterion” is intended to mean an anion or an anionic group derived from an organic or mineral acid which counterbalances the cationic charge of the dye; more particularly, the anionic counterion is chosen from: i) halides such as chloride or bromide; ii) nitrates; iii) sulfonates, including C₁-C₆ alkylsulfonates: Alk-S(O)₂O⁻ such as methylsulfonate or mesylate and ethylsulfonate; iv) arylsulfonates: Ar—S(O)₂O⁻ such as benzenesulfonate and toluenesulfonate or tosylate; v) carboxylates Alk-C(O)—OH with Alk representing a (C₁-C₆)alkyl group optionally substituted with one or more hydroxyl or carboxylate groups such as citrate; vi) succinate; vii) tartrate; viii) lactate; ix) alkyl sulfates: Alk-O—S(O)O⁻ such as methyl sulfate and ethyl sulfate; x) aryl sulfates: Ar—O—S(O)O⁻ such as benzene sulfate and toluene sulfate; xi) alkoxy sulfates: Alk-O—S(O)₂O⁻ such as methoxy sulfate and ethoxy sulfate; xii) aryloxy sulfates: Ar—O—S(O)₂O⁻, xiii) phosphates O═P(OH)₂—O⁻, O═P(O⁻)₂—OH O═P(O⁻)₃, HO—[P(O)(O⁻)]_{w}—P(O)(O⁻)₂ with w being an integer; xiv) acetate; xv) triflate; and xvi) borates such as tetrafluoroborate, and xvii) disulfate (O═)₂S(O⁻)₂ or SO₄²⁻ and monosulfate HSO₄⁻;
urea is the compound of formula CO(NH₂)₂
the term “urea derivative” is intended to mean any compound other than urea itself comprising in its chemical formula a carbonyl group simply bonded to two nitrogen atoms.

As previously explained, the process according to the invention uses i) at least one thiol-based reducing agent and ii) at least one compound of formula (I) as defined previously, it being understood that the composition(s) (A), (B) and (D) is (are) acids; preferably said composition(s) (A), (B) and (D) has (have) a pH of between 1 and 5 inclusive, preferably between 2.5 and 4 inclusive, such as pH=3.5.

The thiol-based reducing agent(s) present in the composition (A) or (D) used according to the invention are chosen from organic compounds comprising one or more mercapto (—SH or —S—) groups, or disulfide (—S—S—) groups, preferably —SH groups, and at least one other function chosen from carboxylic acid, amine, amide, ester and alcohol functions, and mixtures thereof, preferably carboxylic acid functions.

The reducing agent(s) used in the invention are chosen from those of formulae i-1 and i-2, and also the organic or mineral acid or base salts thereof, optical isomers thereof and tautomers thereof, and the solvates such as hydrates:

| R-SH | R′-S-R″ |
|---|---|
| i-1 | i-2 |

in which formulae i-1 and i-2:

R represents:
a linear or branched (C₁-C₈)alkyl group, preferably a linear or branched (C₁-C₆)alkyl group:
a) substituted with one or more groups chosen from carboxy C(O)OH, hydroxyl —OH and thiol —SH, preferably carboxy;
and/or
b) interrupted with one or more heteroatoms or groups chosen from —O—, —S—, —N(R″′)—, C(O) or combinations thereof such as —O—C(O)—, —C(O)— O—, —N(R″′)—C(O)— or —C(O)—N(R″′)—;
a (hetero)aryl optionally substituted with one or more hydroxyl, thiol or carboxy groups, preferably carboxy groups;

R′ and R″, which may be identical or different, represent a (C₁-C₈)alkyl group, preferably a (C₁-C₆)alkyl group, substituted with one or more groups chosen from hydroxyl, thiol and carboxy, preferably carboxy;

or else R′ and R″ form, together with the sulfur atom which bears them, a 5- to 7-membered heterocyclic group, which is preferably saturated, which comprises from 1 to 3 heteroatoms, and which is optionally substituted (in particular with one or more carboxy or (C₁-C₆)alkyl groups optionally substituted with one or more hydroxyl, thiol or carboxy groups, preferably carboxy groups), more preferentially the heterocyclic group is a dithiolane group optionally substituted with a (C₁-C₆)alkyl group optionally substituted with one or more carboxy groups.

The reducing agents are preferably of formula i-1, for which R represents a linear or branched (C₁-C₈)alkyl group, preferably a linear or branched (C₁-C₆)alkyl group, more preferentially a linear or branched (C₁-C₄)alkyl group:

a) substituted with one or more groups chosen from carboxy C(O)OH, hydroxyl —OH and thiol —SH, preferably carboxy;

and/or

b) interrupted with one or more heteroatoms or groups chosen from —O—, —S—, —N(R″′)—, C(O) or combinations thereof such as —O—C(O)—, —C(O)—O—, —N(R″′)—C(O)— or —C(O)—N(R″′)—;

Alternatively, the reducing agents are of formula i-1 for which R represents:

a phenyl group optionally substituted with one or more hydroxyl, thiol or carboxy groups, preferably carboxy groups; or
a 5- to 10-membered, preferably 9- or 10-membered bicyclic, heteroaryl, comprising from 1 to 4 heteroatoms chosen from O, S or N, preferably N, optionally substituted with one or more hydroxyl, carboxy or thiol groups, preferably carboxy groups.

Alternatively, the reducing agents are of formula i-2, in particular those for which R′ and R″, which may be identical or different, represent a (C₁-C₈)alkyl group, preferably (C₁-C₆)alkyl group, more preferentially (C₁-C₄)alkyl group, substituted with one or more groups chosen from hydroxyl, thiol and carboxy, preferably carboxy.

Alternatively, the reducing agents are of formula i-2, in particular those for which R′ and R″ form, together with the sulfur atom which bears them, a 5- to 7-membered heterocyclic group, which is preferably saturated, which comprises from 1 to 3 heteroatoms, and which is optionally substituted with one or more carboxy or (C₁-C₆)alkyl groups optionally substituted with one or more hydroxyl, thiol or carboxy groups, more preferentially the heterocyclic group is a dithiolane group optionally substituted with a (C₁-C₆)alkyl group optionally substituted with one or more hydroxyl, thiol or carboxy groups, preferably carboxy groups.

Preferably, the reducing agent(s) are chosen from thioglycolic acid, thiolactic acid, glutathione, thioglycerol, thiomalic acid, 3-mercaptopropionic acid, thiodiglycol, 2-mercaptoethanol, dithiothreitol, thioxanthine, thiosalicylic acid, thiodiglycolic acid, lipoic acid, N-acetylcysteine, and thioglycolic or thiolactic acid esters and amides, in particular glyceryl monothioglycolate, and mixtures of these compounds.

More preferentially, the reducing agent(s) of the invention are chosen from thiolactic acid, 3-mercaptopropionic acid, thioglycolic acid, and also the organic or mineral salts thereof, preferably organic salts thereof such as the ammonium salts, more preferentially thiolactic acid and ammonium thiolactate.

The thiol-based reducing agent(s) as defined previously may be used especially in the form of organic or mineral salts, in particular alkali metal salts such as sodium and potassium salts, alkaline-earth metal salts, for example magnesium and calcium salts, ammonium salts, amine salts and amino alcohol salts. Ammonium thioglycolate may thus be used as thiol.

The thiol-based reducing agent(s) included in the composition (A) or (D) according to the invention is (are) preferably present in an amount of less than 6% by weight, in particular less than 4%, more particularly less than 2% by weight, more preferentially in an amount of between 0.1% and 6% by weight, even more preferentially in an amount of between 0.2% and 1% by weight, relative to the total weight of the composition (A) or (D) respectively.

The pH of the composition (A) and/or (B) or (D) according to the invention may be adjusted to the desired value by means of basifying agents or acidifying agents that are usually used.

The organic alkaline agent(s) are preferably chosen from alkanolamines, in particular mono-, di- or tri-hydroxy(C₁-C₆)alkylamines, such as 2-amino2-methylpropanol triethanolamine, oxyethylenated and/or oxypropylenated ethylenediamines, amino acids, the polyamines of formula (VIII) below, and mixtures thereof:

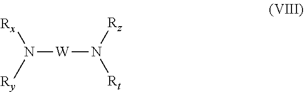

in which formula (VIII) W is a divalent C₁-C₆ alkylene radical optionally substituted with one or more hydroxyl groups or a C₁-C₆ alkyl radical, and/or optionally interrupted with one or more heteroatoms such as O, or NRᵤ; Rₓ, R_{y}, R_{z}, Rₜ and Rᵤ, which may be identical or different, represent a hydrogen atom or a C₁-C₆ alkyl, C₁-C₆ hydroxyalkyl or C₁-C₆ amino alkyl radical.

Examples of amines of formula (VIII) that may be mentioned include 1,3-diaminopropane, 1,3-diamino-2-propanol, spermine and spermidine.

The term “alkanolamine” is intended to mean an organic amine comprising a primary, secondary or tertiary amine function, and one or more linear or branched C₁ to C₈ alkyl groups bearing one or more hydroxyl radicals.

Organic amines chosen from alkanolamines such as monoalkanolamines, dialkanolamines or trialkanolamines comprising one to three identical or different C₁ to C₄ hydroxyalkyl radicals are in particular suitable for performing the invention.

Among the compounds of this type, mention may be made of monoethanolamine (MEA), diethanolamine, triethanolamine, monoisopropanolamine, diisopropanolamine, N,N-dimethylethanolamine, 2-amino-2-methyl-1-propanol, triisopropanolamine, 2-amino-2-methyl-1,3-propanediol, 3-amino-1,2-propanediol, 3-dimethylamino-1,2-propanediol and tris(hydroxymethyl)aminomethane.

More particularly, the amino acids that may be used are of natural or synthetic origin, in their L, D or racemic form, and include at least one acid function chosen more particularly from carboxylic acid, sulfonic acid, phosphonic acid and phosphoric acid functions. The amino acids may be in neutral or ionic form.

Among the basifying agents, mention may more particularly be made of aqueous ammonia, alkanolamines, and mineral or organic hydroxides.

Among the acidifying agents, mention may be made of i) hydrochloric acid HCl, ii) hydrobromic acid HBr, iii) sulfuric acid H₂SO₄, iv) alkylsulfonic acids: Alk-S(O)₂OH, such as methanesulfonic acid and ethanesulfonic acid; v) arylsulfonic acids: Ar—S(O)₂OH, such as benzenesulfonic acid and toluenesulfonic acid; vi) (poly)hydroxylated carboxylic acids, such as citric acid, succinic acid, tartaric acid or lactic acid, vii) alkoxysulfinic acids: Alk-O—S(O )—OH, such as methoxysulfinic acid and ethoxysulfinic acid; viii) aryloxysulfinic acids, such as tolueneoxysulfinic acid and phenoxysulfinic acid; ix) phosphoric acid H₃PO₄; x) acetic acid CH₃C(O)—OH; xi) triflic acid CF₃SO₃H and xii) tetrafluoroboric acid HBF₄; more particularly, the mineral or organic acids used to acidify the composition(s) are chosen from hydrochloric acid, orthophosphoric acid, sulfuric acid, carboxylic acids, for instance acetic acid, tartaric acid, citric acid and lactic acid, and sulfonic acids.

The concentration of pH-adjusting agent(s) is in particular adjusted according to the pH of 1 to 5, preferentially 2.5 to 4, more preferentially pH=3.5 desired for the compositions (A) and/or (B) or (D).

The second ingredient used in the invention is the dicarboxylic acid or derivative of formula (I) as defined previously.

The compounds of formula (I) may be such that just one of the R or R′ groups represents a hydrogen atom, i.e. the compound of formula (I) is a monoacid compound. According to another preferred embodiment, R and R′ represent a hydrogen atom.

Preferably, the compounds of formula (I) are such that R and R′ represent a hydrogen atom, i.e. the compounds of formula (I) are diacid compounds of formula: HO—C(O)-ALK-C(O)—OH and also the mineral or organic salts thereof, in which ALK represents a linear or branched (C₁-C₆)alkylene group.

The compound(s) of formula (I) may be such that the ALK radical represents a methylene, ethylene, n-propylene, n-butylene, n-hexylene, preferably n-propylene group.

The compound(s) of formule (I) may be in an amount of less than 20% by weight, particularly between 1% and 15% by weight, more particularly between 2% and 10% by weight, relative to the total weight of the composition (B) or (D).

According to one preferred embodiment of the invention, the total amount of thiol-based reducing agent(s) i) as defined previously and of carboxylic acid(s) ii) as defined previously is less than 17% by weight relative to the total weight of the compositions (A)+(B) or (D), particularly between 1% and 16% by weight, more particularly between 2% and 14%, even more particularly between 4% and 12%, preferentially between 5% and 11% by weight, relative to the total weight of the compositions (A)+(B) or (D).

The pH of the composition (A) and (B) or (D) is preferably between 1 and 5 inclusive, preferably between 2.5 and 4, more preferentially pH=3.5.

The composition(s) (A) and/or (B) and/or (D) may comprise less than 1% by weight, preferably less than 0.5% by weight of 4,5-dihydroxy-1,3-bis(hydroxymethyl)imidazolidin-2-one, relative to the total weight of the composition (A) or (B) or (D), more preferably the composition(s) (A) and/or (B) and/or (D) may be free of 4,5 -dihydroxy-1,3-bis(hydroxymethyl)imidazolidin-2-one.

The composition(s) (A) and/or (B) and/or (D) may comprise less than 1% by weight, preferably less than 0.5% by weight of urea and derivatives thereof, relative to the total weight of the composition (A) or (B) or (D), more preferably the composition(s) (A) and/or (B) and/or (D) is free of urea and derivatives thereof.

According to one preferred embodiment, the composition(s) used according to the invention are non-dyeing, i.e. they do not comprise any direct dyeing or oxidation dyeing agents other than the non-thiol-based reducing agents when they are coloured.

According to a particular embodiment of the invention, the composition(s) (A) and/or (B) or (D) used in the process according to the invention may also comprise one or more non-ionic, anionic, cationic, or amphoteric or zwitterionic surfactants.

The compositions used in the process according to the invention may thus be in any form that is compatible with application to keratin fibres, for example in the form of a wax, a paste, a more or less fluid or thick cream, a gel, a foam, a spray or a lotion.

The compositions (A), (B) or (D) described previously are applied to wet or dry keratin fibres. The compositions (A) and (B) are applied sequentially, in particular (A) then (B), or simultaneously, preferably simultaneously. Even more preferentially, the process uses the composition (D) as defined previously, applied to dry or wet keratin fibres.

The compositions (A) and/or (B) or (D) are usually left in place on the fibres for a time generally ranging from 1 minute to 1 hour and preferably from 5 minutes to 30 minutes for each composition.

As described previously, the process according to the invention comprises a step iv) of heat treatment of the fibres by means of a heating tool.

This heat treatment step iv) is generally performed following the application of the composition(s) (A), (B) or (D) described above, optionally after removal thereof by rinsing.

Preferably, the heating tool is chosen from a hairstyling hood, a straightening iron (or flat iron), a hairdryer and an infrared-ray dispenser, and more preferentially the heating tool is a straightening iron. The iron is applied by successive separate strokes lasting a few seconds or by gradual movement or sliding along the locks.

The heat treatment step iv) is generally carried out at a temperature ranging from 30 to 250° C., preferably from 60 to 230° C. and more preferentially from 100 to 200° C. for resistant hair and from 60 to 150° C. for brittle hair, such as African hair or caucasian hair that has been bleached.

The process for treating keratin fibres may comprise, as heat treatment iv), a step of straightening/uncurling the keratin fibres by means of a heating tool chosen from irons and a steam iron, i.e. “irons” which comprise a device that emits steam and that applies this steam before, during or after the straightening/uncurling.

For the purposes of the present invention, the term “iron” is intended to mean a device for heating keratin fibres by placing said fibres in contact with the heating device. The end of the iron which comes into contact with the keratin fibres generally has two flat surfaces. These two surfaces may be made of metal or of ceramic. In particular, these two surfaces may be smooth or crimped or curved.

The iron or the steam iron may be at a temperature of between 65° C. and 250° C., particularly between 80° C. and 230° C., more particularly greater than or equal to 100° C. and preferentially between 100° C. and 190° C. Preferably, the heat treatment step of the process for treating keratin fibres is performed at a temperature ranging from 150° C. to 230° C., preferably ranging from 160° C. to 230° C., preferentially ranging from 160° C. to 210° C., especially ranging from 180° C. to 200° C.

As examples of irons that may be used in the straightening process according to the invention, mention may be made of any type of flat iron with steam, and in particular, in a non-limiting manner, those described in patents U.S. Pat. Nos. 5,957,140 and 5,046,516.

The steam iron may be applied by successive separate strokes lasting a few seconds or by gradual movement or sliding along the locks of keratin fibres, especially of hair.

Preferably, the steam iron is applied in the process according to the invention by a continuous movement from the root to the end of the hair, in one or more passes, in particular in two to twenty passes. The duration of each pass of the steam iron may range from 2 seconds to 1 minute.

Advantageously, steam is applied to the keratin fibres, especially the hair, at a flow rate of less than 5 g/min, especially of between 1 and 4 g/min.

Steam may be applied using any device known per se for generating the amount of steam of use in the process of the invention. According to a particular embodiment, this machine is portable, i.e. the tank for generating steam is in contact with the part of the device comprising the steam-dispensing orifices.

The steam application step may be performed before, during or after the heating step, and preferably before.

Preferably, the step iv) of straightening/uncurling the keratin fibres is performed for a time that may range from 2 seconds to 30 minutes, preferentially from 2 seconds to 20 minutes, better still from 2 seconds to 10 minutes, better still from 2 seconds to 5 minutes and even better still from 2 seconds to 2 minutes.

Preferably, step iv) is performed with a steampod steam straightening device.

The process according to the invention may also comprise an additional step of drying the keratin fibres, after the steps i) and ii) of application of the composition (A) and (B) or the step iii) of application of the composition (D), optionally one or more rinsing steps, and before the heat treatment step iv). The drying step may be performed using a hairdryer or a drying hood or naturally. The drying step is advantageously carried out at a temperature ranging from 20° C. to 70° C.

The term “rinsing step” is intended to mean step of rinsing with water.

After the step iv) of straightening/uncurling using the steam iron, the keratin fibres may be optionally rinsed or washed with a shampoo. The keratin fibres are subsequently optionally dried using a hairdryer or a drying hood or naturally.

The process according to the invention may be performed on natural keratin fibres, especially natural hair.

The process according to the invention may be performed on keratin fibres, especially damaged hair. The term “damaged hair” is intended to mean dry or coarse or brittle or split or limp hair.

The process of the invention is particularly performed on sensitized human keratin fibres, especially hair, such as bleached, relaxed or permanent-waved fibres.

The process according to the invention may be performed on keratin fibres, especially hair, which is wet or dry. Preferentially, the process is performed on natural keratin fibres.

After the steps of applying the compositions (A) and/or (B) or (D) of the process of the invention, and before carrying out the heat treatment step iii), the composition(s) A and/or (B) or (D) applied may be left on for a period ranging from 1 to 60 minutes, preferably ranging from 2 to 50 minutes, preferentially ranging from 5 to 45 minutes. The leave-on period may take place at a temperature ranging from 15° C. to 45° C., preferably at ambient temperature (25° C.).

The cosmetic compositions (A) and (D) as described previously are advantageously applied to the keratin fibres in an amount ranging from 0.1 to 10 grams and preferably from 0.2 to 5 grams of composition (A) or (D), respectively, per gram of keratin fibres.

The cosmetic composition (B) as described previously is advantageously applied to the keratin fibres in an amount ranging from 0.1 to 10 grams and preferably from 0.2 to 5 grams of composition (B) per gram of keratin fibres.

After application of the cosmetic composition(s) (A) and/or (B) or (D) to the keratin fibres, said fibres may be wrung out to remove the excess composition or washed with water.

According to one embodiment of the invention, the process for relaxing the curls and/or for straightening keratin fibres of the invention comprises:

iii) the application to said fibres of an acidic composition (D) as defined previously, preferably having a pH of between 1 and 5 inclusive, and containing:

one or more thiol-based reducing agents in an amount of less than 8% by weight relative to the total weight of the composition (D) and
one or more dicarboxylic acids of formula (I):
HO—C(O)-ALK-C(O)—OH and also the mineral or organic salts thereof, in which ALK represents a linear or branched (C₁-C₆)alkylene group; and

iv) a step of heat treatment of the fibres by means of a heating tool;

it being understood that step iv) is carried out after step iii).

The treatment process according to the invention may be performed before, during and/or after an additional process of cosmetic treatment of the keratin fibres, such as a process for temporary curl relaxation (relaxing of curls with curlers, a crimping iron or a straightening iron) or a process for long-lasting curl relaxation (permanent-waving or uncurling) of the keratin fibres.

The process according to the invention may also comprise an additional step of partial predrying of the hair fibres, before the step of increasing the temperature. This predrying step may be performed, for example, by means of a dryer or of a drying hood or else by drying naturally.

Before and/or on conclusion of the process according to the invention, the keratin fibres may optionally be washed with a shampoo, followed by rinsing with water, before being dried or left to dry.

Preferably in this embodiment, the process comprises:

1) the application to the fibres of a composition (A) as defined previously,

2) the application to the fibres of a composition (B) as defined previously,

3) preferably a period of waiting, preferably for between 5 and 60 minutes,

4) preferably a rinsing step,

5) then a step of drying at ambient temperature or by heat treatment, preferably by blow drying,

6) then a step of heat treatment of the fibres by means of a heating tool, with or without pulling effect by means of a brush or comb, preferably with flat irons, a heating comb,

7) then optionally a step of washing the keratin fibres,

8) then optionally an oxidizing treatment, such as hydrogen peroxide, preferably in an amount of between 0.3% and 12% by weight (1 to 40 volumes) and more preferentially between 0.6% and 2% by weight (2 to 7 volumes).

Preferably, the compositions (A) and (B) are applied together, or else a composition (D) as defined previously is applied to the fibres. According to one advantageous variant, the process comprises:

1) the application to the fibres of a composition (D) as defined previously,

2) then preferably a period of waiting, preferably for between 5 and 60 minutes,

3) preferably a rinsing step,

4) then a step of drying at ambient temperature or by heat treatment, with or without pulling effect by means of a brush or comb, preferably by blow drying,

5) then a step of heat treatment of the fibres by means of a heating tool, preferably with flat irons, a heating comb,

6) then optionally a step of washing the keratin fibres, and

7) then optionally an oxidizing treatment, such as hydrogen peroxide, preferably in an amount of between 0.3% and 12% by weight (1 to 40 volumes) and more preferentially between 0.6% and 2% by weight (2 to 7 volumes).

In the case where it is desired to treat brittle hair, step 5 can be avoided or replaced with a heat treatment or blow drying.

The process according to the invention may be repeated so as to increase the effects, until the desired level of straightening is obtained. However, and this constitutes an advantage of the present invention, from the very first implementation of the process according to the invention, including implementation without placing the hair under tension, a substantial reduction in the volume of the head of hair is observed. When the hair is curly, relaxation of the curls and/or better curl definition are also observed.

It is also found that the unpleasant odours that are given off during the straightening process, or that remain on the straightened hair, are reduced by means of performing the process according to the invention.

According to a preferred embodiment, the process is repeated several times, either consecutively or after a delay ranging from a few hours to a few days. Depending on the situation, it is possible to carry out a series of treatments, in which the result of the first determines the one that follows. For example, the series begins with a first treatment in which the conditions are limited, for example through the use of a composition with a low concentration, or through the use of a moderate heat. Then, depending on the result of this first treatment, and the judgement made by the user, the customer or the hairstylist, it is possible to modulate the subsequent treatment and so on.

According to one preferred embodiment, the process of the invention is applied to keratin fibres chosen from the hair, in particular natural Brazilian hair, in particular of type 3A, and kinky keratin fibres, in particular kinky hair.

A subject of the invention is also the use of the composition (A) with (B), or (D)as defined previously, combined with a heat treatment of the fibres by means of a heating tool, for curl-relaxing or straightening keratin fibres, in particular on keratin fibres chosen from the hair, in particular natural Brazilian hair, in particular of type 3A, and kinky keratin fibres, in particular kinky hair.

For the purposes of the invention, the hair types are classified according to the André Walker classification. This typology makes it possible to identify the various types of hair (from 1 to 4) and also the thickness thereof (from a to c). According to the André Walker analysis grid, the hair is divided up into four types. 1=straight hair, 2=wavy hair, 3=curly and frizzy hair and 4=kinky hair.

For hair 1 to hair 3, the general appearance is observed visually in order to determine the category thereof. For hair of type 4, kinky hair, the head of hair is wetted with water and wrung dry, and the way in which the hair curls or does not curl is observed visually.

It is thus possible to categorize the hair as follows:

Type 1: Straight hair

Type 2: Wavy hair

Type 2A hair is slightly wavy in the shape of an “S”. The hair falls and is close to the head. It is hair that is rather shiny, and that has a tendency to be fine and quite easy to handle. Type 2B is wavy hair in the shape of an “S”. It is hair that is slightly unruly on the edges, tending to become frizzy.

Type 2C is wavy hair that is much thicker and with obvious curls. It frizzes very easily and can become difficult to style.

Type 3: Curly hair

Type 3A hair forms “S”-shaped curls that are very well defined and supple. The curls are generally large and the head of hair shiny.

Type 3B hair forms well-defined curls which can be frizzy or else corkscrew-shaped.

Type 3C forms tight corkscrew curls with the circumference of a pencil or a straw. The texture may be kinky or very curly.

Type 4: Kinky hair

The first type of kinky hair is 4A. The hair is clearly curly/frizzy and this is the least dry of the kinky range. The very tight frizz is like small springs.

Type 4B does not form “S”-shaped curls, but instead “Z”-shaped spirals. These are very pronounced waves. The end of the hair can sometimes itself form a small “S”-shaped curl. It is this kinky hair which resembles a cotton cloud when it is disentangled: it is soft and malleable if well maintained. Hair 4B decreases on average down to 75% of its actual, i.e. relaxed, length.

Type 4C is kinky but there is no trace of curls. It is the driest hair that exists. It is hair that is sometimes fine, but often very thick. It shrinks to more than 75% of its actual length and in general does not show any curls or frizz.

A subject of the present invention is also a kit that is suitable for performing the process of the invention. This kit comprises at least two compartments:

a first compartment comprising a composition (A) as defined previously,

a second compartment comprising a composition (B) as defined previously.

The compositions of this kit are packaged in separate compartments, which may be optionally accompanied by suitable identical or different application means, such as fine brushes, coarse brushes or sponges.

The abovementioned kit may also be equipped with means for dispensing the desired mixture on the hair, for instance the device described in patent FR 2 586 913.

The examples that follow illustrate the present invention, and should not in any way be considered as limiting the invention. All percentages are weight percentages unless specifically stated otherwise.

### EXAMPLES

The following compositions were prepared and then applied to type 3A Brazilian hair:

| | | TLA | Glutaric | | Lock length (in |
|---|---|---|---|---|---|
| | | (% by | acid (% by | | cm) after |
| compositions | | weight) | weight) | Water | treatment |
| 1 | control | 0 | 10 | qs 100 | 21 |
| 2 | comparative | 0.5 | 0 | qs 100 | 21 |
| 3 | invention | 0.5 | 10 | qs 100 | 24 |
| 4 | comparative | 1 | 0 | qs 100 | 22 |
| 5 | invention | 1 | 10 | qs 100 | 25 |
| 6 | comparative | 2 | 0 | qs 100 | 22 |
| 7 | invention | 2 | 10 | qs 100 | 25 |
| 8 | comparative | 4 | 0 | qs 100 | 22 |
| 9 | invention | 4 | 10 | qs 100 | 25 |
| 10 | comparative | 6 | 0 | qs 100 | 22 |
| 11 | invention | 6 | 10 | qs 100 | 25 |

The pH of compositions 1 to 11 was adjusted to pH=3.5 with 2-amino-2-methylpropanol.

FIG. 1 effect of the decrease in frizziness as a function of the TLA concentration with and without the addition of glutaric acid (10=maximum effectiveness/0=minimal effectiveness)

The effectiveness of the treatment as a function of the concentration of thiolactic acid (from 0.5% to 8%), with and without the addition of 10% glutaric acid, was thus compared.

It emerges from the comparative tests above that, even with a very low concentration of thiol-based reducing agent, such as thiolactic acid (TLA), it is possible to modify the shape of the hair, making natural Brazilian hair of type 3A straight.

In addition, the odour of the composition is very tolerable or even odourless. Indeed, it emerges from the tests above that the application of the composition comprising a low concentration of thiol-based reducing agent (1% of TLA) and of compound of formula (I) (10% of glutaric acid), makes it possible to obtain a performance equivalent to that of a composition comprising a much higher concentration (8 times higher) of thiol-based reducing agent (8% TLA), this being with a much weaker odour.

### Other Comparative Tests

Protocols

| | | |
|---|---|---|
| A | 1% TLA + Glutaric acid pH 3.5 | Protocol with flat iron |
| B | 8% TLA pH 3.5 | Protocol with flat iron |

The quality of the hair is noted on the basis of a visual evaluation of the hair after treatment, washing and drying:

| | Condition of the fibre | After 2 | After 3 |
|---|---|---|---|
| Protocol | after 1 application | applications | applications |
| A | 10 | 9 | 8 |
| B | 10 | 7 | 5 |

Curl relaxing can be carried out to obtain effects such as easier combing.

| | | Glutaric | Heating | |
|---|---|---|---|---|
| Type of hair | TLA | Acid | conditions | Result |
| frizzy 3A | 1% | 10% | Irons | Total straightening |
| | | | | in one application |
| frizzy 3A | 0.5% | 5% | Irons | Curl relaxing after |
| | | | | one application. |
| | | | | Total straightening |
| | | | | in 2 applications |
| frizzy 3A | 0.2% | 2% | Irons | Curl relaxing after |
| | | | | one application. |
| | | | | Total straightening |
| | | | | in 3 applications |
| Kinky hair 4 | 0.2% | 2% | Irons | Curl relaxing after |
| | | | | one application |
| | | | | enabling easier |
| | | | | combing |
| Kinky hair 4 | 0.2% | 2% | Heating comb | Easier combing |
| Kinky hair 4 | 0.4% | 4% | Heating comb | Curl relaxing after |
| | | | | one application |
| | | | | enabling easier |
| | | | | combing |
| Kinky hair 4 | 1% | 10% | Hair dryer | Curl relaxing after |
| | | | | one application |
| | | | | enabling easier |
| | | | | combing |

It emerges from the comparative tests above that, even with a very low concentration of thiol-based reducing agent, such as thiolactic acid, it is possible to modify the shape of the hair, making natural Brazilian hair 3A straight, or even making kinky hair 4 straight.

### Additional Comparative Tests

The following compositions were prepared. The pH of the compositions was measured using a pH meter.

| Ingrèdients | Composition 12 | Composition 13 | Composition 14 |
|---|---|---|---|
| Glutaric acid | 15% | 15% | 15% |
| Cysteine hydrochloride | 5% | 0 | 0 |
| Thiolactic acid | 0 | 4.37% | 4.37% |
| 4,5-dihydroxy-1,3- | 10% | 10% | 0 |
| bis(hydroxymethyl) | | | |
| imidazolidin-2-one | | | |
| Water | q.s.p. 100% | q.s.p. 100% | q.s.p. 100% |
| pH | 1.11 | 2.03 | 1.7 |

Each of the compositions have been tested according to the following test protocol.

### Test Protocol

5.4 g of compositions 12 to 14 were applied homogeneously using a hair coloring brush to Brazilian type 4 natural hair swatches (2.7 g hair swatches) positioned on a heating plate covered with an aluminium foil and maintained at a temperature of 27° C. The swatches were left to stand for 30 minutes, drained with absorbent paper and then rinsed with water while massaging the swatches 10 times between two fingers. The swatches were then blow-dried 10 times at 80° C. and straighten 10 times (6s each time) using a hair straightener maintained at a temperature of 230° C. The swatches were then shampooed (0.4 g of shampoo/g of hair).

### Results

For each sample, the length of the swatch, the width of the swatch (measured at half height) and the number of remaining curls were measured.

| | | | Number of |
|---|---|---|---|
| | Length of | Width of | remaining |
| Type of hair | swatch (cm) | swatch (cm) | curls |
| Hair swatch without | 21.8 | 4 | 5 |
| treatment (comparative) | | | |
| Hair swatch treated | 24 | 1 | 7 |
| with composition 12 | | | |
| (comparative) | | | |
| Hair swatch treated | 26.5 | 2 | 3 |
| with composition 13 | | | |
| Hair swatch treated | 27 | 2.5 | 0 |
| with composition 14 | | | |

As can be seen from the above results, the process according to the present invention provides good performances in terms of the straightening of keratin fibres. The obtained result is even better when the composition does not comprise 4,5-dihydroxy-1,3-bis(hydroxymethyl)imidazolidin-2-one.

1.-20. (canceled)

21. A method for relaxing and/or straightening keratin fibres comprising:
i) simultaneously or sequentially applying to the keratin fibres a composition (A) and a composition (B); or
ii) alternatively, applying to the keratin fibres a composition (D); and
iii) heating the keratin fibres with a heating tool after the composition (A) and the composition (B) are applied to the keratin fibres, or after the composition (D) is applied to the keratin fibres;
wherein the composition (A) is acidic and comprises at least one thiol-based reducing agent in an amount of less than 8% by weight relative to the total weight of the composition (A);
wherein the composition (B) comprises at least one compound chosen from compounds of formula (I): (I) RO—C(O)-ALK-C(O)—OR′, or mineral or organic salts thereof;
wherein in formula (I):
ALK represents a linear or branched (C₁-C₆)alkylene group;
R and R′, which may be identical or different, are chosen from a hydrogen atom, an optionally substituted (C₁-C₆)alkyl group, an optionally substituted (C₂-C₆)alkenyl group, an optionally substituted (hetero)aryl(C₁-C₄)alkyl group, an optionally substituted (hetero)cyclic(C₁-C₄)alkyl group, or an optionally substituted (hetero)aryl group, or combinations thereof;
wherein the composition (D) is acidic and comprises:
a) at least one thiol-based reducing agent in an amount of less than 8% by weight, relative to the total weight of the composition (D); and
b) at least one compound chosen from the compounds of formula (I) defined above, or mineral or organic salts thereof;
wherein the at least one thiol-based reducing agent comprised in composition (A) and composition (D) is chosen from compounds of formula i-1, formula i-2, organic or mineral acid or base salts thereof, optical isomers thereof, tautomers thereof, solvates thereof, or combinations thereof:

| R-SH | R′-S-R″ |
|---|---|
| i-1 | i-2 |

wherein in formulae i-1 and i-2:
R represents:
a linear or branched (C₁-C₈)alkyl group:
a) substituted with one or more groups chosen from carboxy C(O)OH, hydroxyl —OH, thiol —SH, or combinations thereof; and/or
b) interrupted with one or more heteroatoms or groups chosen from —O—, —S—, —N(R″′)—, C(O), or combinations thereof;
a (hetero)aryl group optionally substituted with one or more groups chosen from hydroxyl group, thiol group, carboxy group, or combinations thereof;
R′ and R″, which may be identical or different, represent a (C₁-C₈)alkyl group, substituted with one or more groups chosen from hydroxyl group, thiol group, carboxy group, or combinations thereof; alternatively R′ and R″ form, together with the sulfur atom which bears the R′ and R″, a 5- to 7-membered heterocyclic group, which comprises from 1 to 3 heteroatoms, and which is optionally substituted with one or more groups chosen from hydroxyl group, thiol group, carboxy group, or combinations thereof.

22. The method of claim 21, wherein the pH of the composition (A) ranges from 1 and 5 and the pH of the composition (D) ranges from 1 and 5.

23. The method of claim 21, wherein the at least one thiol-based reducing agent comprises at least one carboxy group.

24. The method of claim 21, wherein the at least one thiol-based reducing agent is chosen from compounds of formula i-1, wherein in formula i-1, R represents a linear or branched (C₁-C₆)alkyl group:
a) substituted with one or more groups chosen from hydroxyl —OH, thiol —SH group, carboxy group, or combination thereof; and/or
b) interrupted with one or more heteroatoms or groups chosen from —O—, —N(R″′)—, C(O), or combinations thereof.

25. The method of claim 21, wherein the at least one thiol-based reducing agent is chosen from thioglycolic acid, thiolactic acid, glutathione, thioglycerol, thiomalic acid, 3-mercaptopropionic acid, thiodiglycol, 2-mercaptoethanol, dithiothreitol, thioxanthine, thiosalicylic acid, thiodiglycolic acid, lipoic acid, N-acetylcysteine, thioglycolic or thiolactic acid esters and amides, or mixtures of thereof.

26. The method of claim 21, wherein the at least one thiol-based reducing agent is present in an amount of less than 6% by weight, relative to the total weight of the composition (A) or the composition (D).

27. The method of claim 21, wherein in formula (I), ALK represents a methylene, an ethylene group, an n-propylene group, an n-butylene group, or an n-hexylene group.

28. The method of claim 21, wherein the at least one compound chosen from compounds of formula (I) is present in an amount of less than 20% by weight, relative to the total weight of the composition (B) or the composition (D).

29. The method of claim 21, wherein the total amount of the at least one thiol-based reducing agent and the at least one compound chosen from compounds of formula (I) is less than 17% by weight, relative to the total weight of the combination of the composition (A) and the composition (B), or the total weight of the composition (D).

30. The method of claim 21, wherein the composition (A), the composition (B), and the composition (D) each comprises less than 1% by weight of 4,5-dihydroxy-1,3-bis(hydroxymethyl)imidazolidin-2-one, relative to the total weight of the composition (A), the composition (B), and the composition (D), respectively.

31. The method of claim 21, wherein the composition (A), the composition (B), and the composition (D) are free of 4,5-dihydroxy-1,3-bis(hydroxymethyl)imidazolidin-2-one.

32. The method of claim 21, wherein the composition (A), the composition (B), or the composition (D) comprises urea or derivatives thereof in an amount less than 1% by weight, relative to the total weight of the composition (A), the composition (B), or the composition (D), respectively.

33. The method of claim 21, wherein the composition (A), the composition (B), and the composition (D) are free of urea or derivatives thereof.

34. The method of claim 21, further comprising:
rinsing the keratin fibres after applying the composition (A) and the composition (B), or after applying the composition (D), and before heating the keratin fibres.

35. The method of claim 21, wherein the heating tool is chosen from a hairstyling hood, a straightening iron, a hairdryer, a heating comb, an infrared-ray dispenser, or a combination thereof.

36. The method of claim 21, wherein the keratin fibres are heated to a temperature ranging from 30° C. to 250° C.

37. The method of claim 21, when the composition (D) is applied to keratin fibres, further comprising:
leaving the composition (D) on the keratin fibres for a period of 5 to 60 minutes;
rinsing the composition (D) from the keratin fibres;
after rinsing, drying the keratin fibres at ambient temperature or blow drying the keratin fibres before heating the keratin fibres with a heating tool, wherein the heating tool is chosen from a hairstyling hood, a straightening iron, a hairdryer, a heating comb, or an infrared-ray dispenser, or a combination thereof;
optionally washing the keratin fibres after heating the keratin fibres; and
optionally performing an oxidizing treatment to the keratin fibres after washing the keratin fibres.

38. The method of claim 21, wherein the keratin fibres are hair.

39. A composition comprising:
at least one thiol-based reducing agent present in an amount of less than 8% by weight, relative to the total weight of the composition, the at least one thiol-based reducing agent being chosen from compounds of formula i-1, formula i-2, organic or mineral acid or base salts thereof, optical isomers thereof, tautomers thereof, solvates thereof, or combinations thereof:

| R-SH | R′-S-R″ |
|---|---|
| i-1 | i-2 |

wherein in formulae i-1 and i-2:
R represents:
a linear or branched (C₁-C₈)alkyl group:
a) substituted with one or more groups chosen from carboxy C(O)OH, hydroxyl —OH, thiol —SH, or combinations thereof; and/or
b) interrupted with one or more heteroatoms or groups chosen from —O—, —S—, —N(R″′)—, C(O), or combinations thereof;
a (hetero)aryl group optionally substituted with one or more groups chosen from hydroxyl group, thiol group, carboxy group, or combinations thereof;
R′ and R″, which may be identical or different, represent a (C₁-C₈)alkyl group, substituted with one or more groups chosen from hydroxyl group, thiol group, carboxy group, or combinations thereof; alternatively, R′ and R″ form, together with the sulfur atom which bears the R′ and R″, a 5- to 7-membered heterocyclic group, which comprises from 1 to 3 heteroatoms, and which is optionally substituted with one or more groups chosen from hydroxyl group, thiol group, carboxy group, or combinations thereof;
and
at least one compound chosen from compounds of formula (I): (I) RO—C(O)—ALK-C(O)—OR′, or mineral or organic salts thereof;
wherein in formula (I):
ALK represents a linear or branched (C₁-C₆)alkylene group;
R and R′, which may be identical or different, are chosen from hydrogen atom, an optionally substituted (C₁-C₆)alkyl group, an optionally substituted (C₂-C₆)alkenyl group, an optionally substituted (hetero)aryl(C₁-C₄)alkyl group, an optionally substituted (hetero)cyclic(C₁-C₄)alkyl group, or an optionally substituted (hetero)aryl group, or combinations thereof;
wherein the composition is acidic.

40. A kit comprising:
a first compartment comprising a composition (A);
a second compartment comprising a composition (B);
wherein the composition (A) is acidic and comprises at least one thiol-based reducing agent in an amount of less than 8% by weight, relative to the total weight of the composition (A), the at least one thiol-based reducing agent being chosen from compounds of formula i-1, formula i-2, organic or mineral acid or base salts thereof, optical isomers thereof, tautomers thereof, solvates thereof, or combinations thereof:

| R-SH | R′-S-R″ |
|---|---|
| i-1 | i-2 |

wherein in formulae i-1 and i-2:
R represents:
a linear or branched (C₁-C₈)alkyl group:
a) substituted with one or more groups chosen from carboxy C(O)OH, hydroxyl —OH, thiol —SH, or combinations thereof; and/or
b) interrupted with one or more heteroatoms or groups chosen from —O—, —S—, —N(R″′)—, C(O), or combinations thereof;
a (hetero)aryl group optionally substituted with one or more groups chosen from hydroxyl group, thiol group, carboxy group, or combinations thereof;
R′ and R″, which may be identical or different, represent a (C₁-C₈)alkyl group, substituted with one or more groups chosen from hydroxyl group, thiol group, carboxy group, or combinations thereof;
alternatively, R′ and R″ form, together with the sulfur atom which bears the R′ and R″, a 5- to 7-membered heterocyclic group, which comprises from 1 to 3 heteroatoms, and which is optionally substituted with one or more groups chosen from hydroxyl group, thiol group, carboxy group, or combinations thereof; and
wherein the composition (B) comprises at least one compound chosen from compounds of formula (I): (I) RO—C(O)-ALK-C(O)—OR′, or mineral or organic salts thereof;
wherein in formula (I):
ALK represents a linear or branched (C₁-C₆)alkylene group;
R and R′, which may be identical or different, are chosen from a hydrogen atom, an optionally substituted (C₁-C₆)alkyl group, an optionally substituted (C₂-C₆)alkenyl group, an optionally substituted (hetero)aryl(C₁-C₄)alkyl group, an optionally substituted (hetero)cyclic(C₁-C₄)alkyl group, or an optionally substituted (hetero)aryl group, or combinations thereof.
